(19) United States
(12) Patent Application Publication
(10) Pub. No.: US 2017/0021108 A1
(43) Pub. Date: Jan. 26, 2017

(54) ANTI-REFLUX SYRINGE ASSEMBLY

(71) Applicant: APTAR STELMI SAS, Villepinte (FR)

(72) Inventors: Mickael SWAL, Chauconin Neufmontiers (FR); Ghislain FOURNIER, La Rochelle (FR)

(73) Assignee: APTAR STELMI SAS, Villepinte (FR)

(21) Appl. No.: 15/300,946

(22) PCT Filed: Apr. 2, 2014
(86) PCT No.: PCT/FR2014/050790

§ 371 Date: Sep. 30, 2016

(51) Int. Cl. A61M 5/315

(52) U.S. Cl. CPC A61M 5/31513; A61M 2005/31516; A61M 2005/31521; A61M 2005/31523; A61M 2207/00

(57) ABSTRACT

An anti-reflux syringe assembly having a reservoir for containing a fluid, the reservoir having a cylindrical side wall, an open distal end, and a proximal end having a proximal end wall provided at its axial center with a through opening; a piston slidable in leaktight manner in the reservoir, the piston having a distal end adapted to connect to a plunger passing through the open distal end of the reservoir, and a proximal end having a proximal end wall. The proximal end walls of the reservoir and of the piston are conical, and the proximal end wall of the reservoir forms an angle α and the proximal end wall of the piston forming an angle β, with 3°<α−β<5°.

The present invention relates to an anti-reflux syringe assembly.

Anti-reflux syringe assemblies are known in the prior art, in particular from Documents WO 2004/033006, WO 2005/032628, WO 2008/151239, WO 2008/151241, and WO 2011/126764. Those devices present certain drawbacks. Thus, in order to prevent two or more pistons sticking together during a storage stage, prior to the pistons being assembled in the reservoirs, projecting profiles are generally provided such as ribs on the proximal end faces of the pistons, which are the faces that come into contact with the fluid. Those projecting profiles serve to prevent the suction cup effect that might otherwise occur when the proximal end face of one piston co-operates in leaktight manner with the distal end of another piston. In contrast, the presence of projecting profiles on the proximal end face of the piston impedes minimizing the dead volume that remains between the piston and the reservoir after actuation, and also makes it impossible to guarantee that the piston closes the outlet from the reservoir in leaktight manner after actuation, i.e. impossible to guarantee the anti-reflux function.

An object of the present invention is to provide an anti-reflux syringe assembly that does not reproduce the above-mentioned drawbacks.

In particular, an object of the present invention is to provide an anti-reflux syringe assembly that is reliable in use and that serves to minimize the dead volume that remains between the piston and the reservoir after actuation.

Another object of the present invention is to provide anti-reflux syringe assemblies for which the risks of pistons sticking together during a stage in which said pistons are in storage, prior to being incorporated in said assemblies, are limited or indeed eliminated.

More particularly, an object of the present invention is to provide an anti-reflux syringe assembly that is simple and inexpensive to fabricate, to store, and to assemble.

The present invention thus provides an anti-reflux syringe assembly comprising:
a reservoir for containing a fluid, said reservoir having a cylindrical side wall, an open distal end, and a proximal end having a proximal end wall provided at its axial center with a through opening; and
a piston slidable in leaktight manner in said reservoir, said piston having a distal end adapted to connect to a plunger passing through said open distal end of the reservoir, and a proximal end having a proximal end wall; and
said proximal end walls of the reservoir and of the piston being conical, said proximal end wall of the reservoir forming an angle α and said proximal end wall of the piston forming an angle β, with

3°+<α−β<5°

Advantageously, α−β=4°.

Advantageously, α=145° (±1°) and β=141° (±1°).

Advantageously, the outside surface of said proximal end wall of the piston in contact with the fluid has an annular zone of surface that is modified, in particular by graining.

Advantageously, said modified annular zone does not include the tip of said proximal end wall of the piston, which tip, after actuation, co-operates in leaktight manner with said through opening in said proximal end wall of the reservoir.

Advantageously, the distal end of the piston has a distal end surface provided with at least one axially projecting profile.

Advantageously, said piston has an inside screw thread adapted to screw onto a corresponding outside screw thread provided on said plunger, said piston being screwed on said plunger.

These characteristics and advantages and others of the present invention appear more clearly from the following detailed description of an advantageous embodiment thereof, made with reference to the accompanying drawings, which are given by way of non-limiting example, and in which:

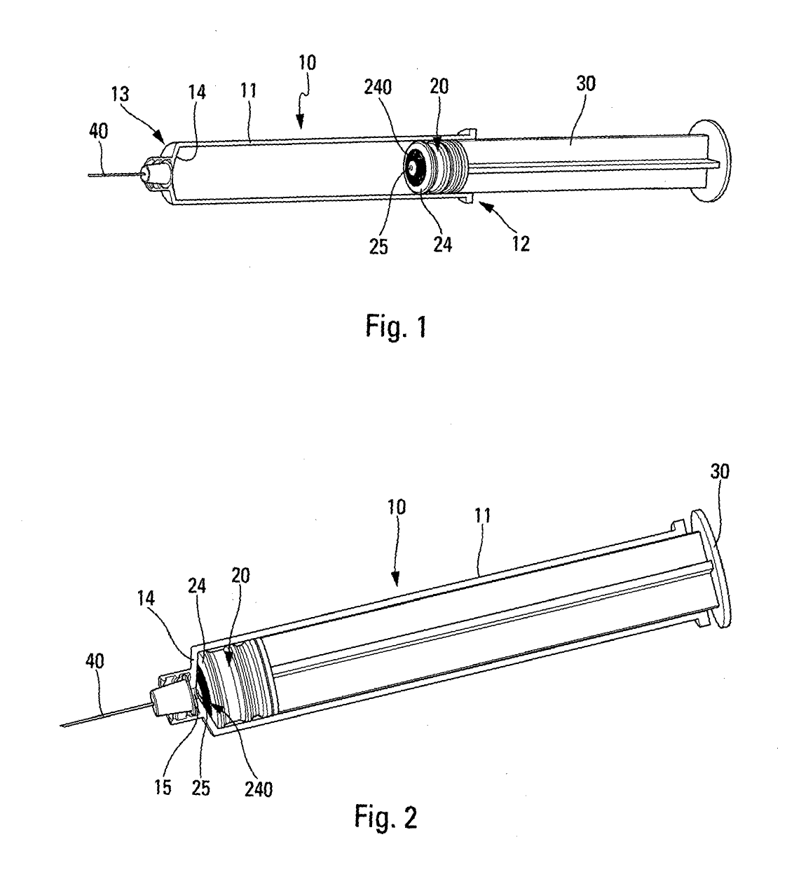

FIG. 1 is a cutaway diagrammatic view of a syringe assembly in an advantageous embodiment of the present invention, prior to actuation;

FIG. 2 is a view similar to FIG. 1, after actuation;

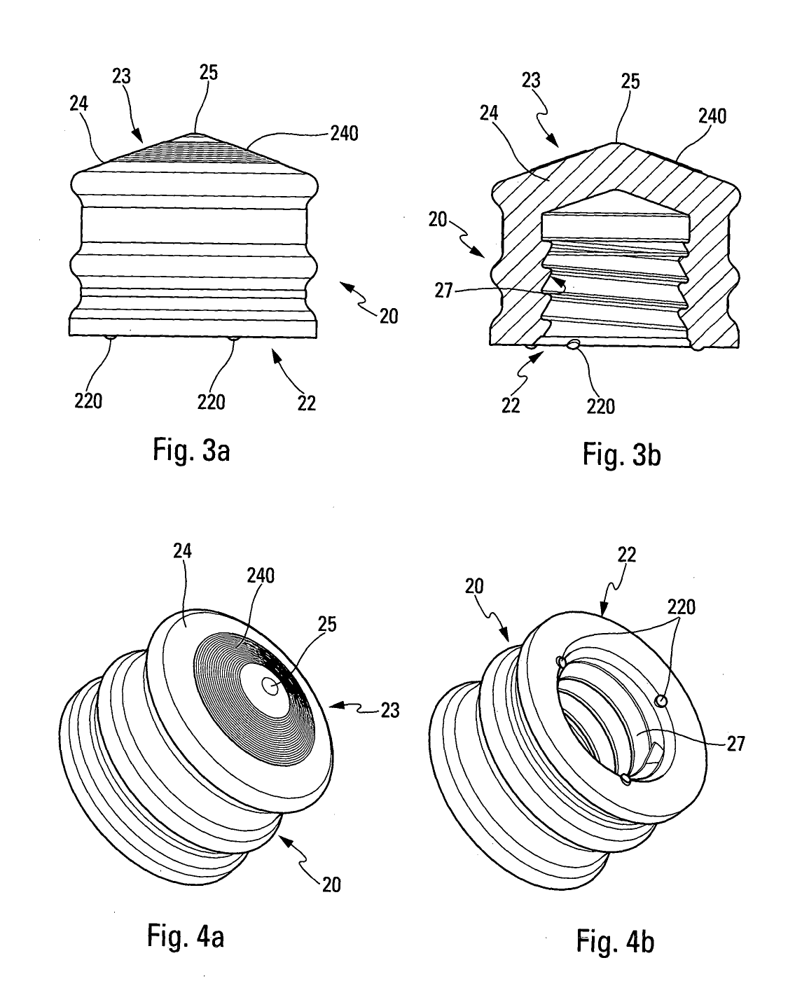

FIG. 3a is a diagrammatic side view of a piston in an advantageous embodiment of the present invention;

FIG. 3b is a longitudinal section view of the FIG. 3a piston;

FIG. 4a is a perspective view of the front of the piston of FIGS. 3a and 3b;

FIG. 4b is a view similar to the view of FIG. 4a, seen from behind;

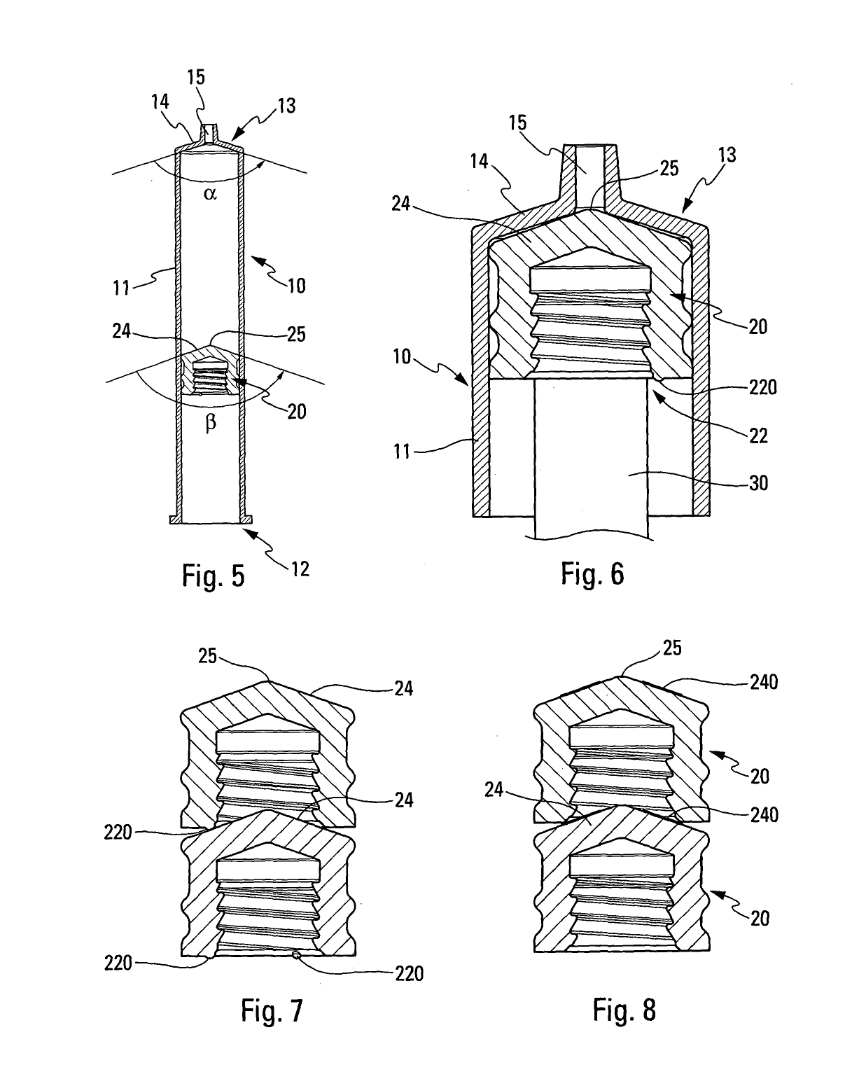

FIG. 5 is a diagrammatic longitudinal section view of a portion of a syringe assembly in an advantageous embodiment of the present invention, prior to use;

FIG. 6 is a detailed longitudinal section view of the FIG. 5 syringe assembly, after use; and

FIGS. 7 and 8 are diagrammatic views of two pistons co-operating with each other during storage, prior to being assembled in a syringe assembly.

With reference to FIGS. 1, 2, 5, and 6, there is shown an anti-reflux syringe assembly in an advantageous embodiment of the invention.

The syringe assembly comprises a reservoir 10 for containing a fluid, a piston 20 slidable in leaktight manner in said reservoir 10, being connected to a plunger 30 surface to expel the fluid contained in said reservoir through a needle 40 fastened to the proximal end 13 of the reservoir 10.

Said reservoir has a cylindrical side wall 11, an open distal end 12, and a proximal end 13 having a proximal end wall 14 provided at its axial center with a through opening 15. The needle 40 is fastened in any appropriate manner to said proximal end 13 of the reservoir in order to connect to said through opening 15.

Said piston 20, sown in detail in FIGS. 3a, 3b, 4a, and 4b, has a distal end 22 and a proximal end 23 including a proximal end wall 24. Said proximal end wall 24 of the piston has an axial tip 25 adapted to co-operate in leaktight manner, after actuation, with said through opening 15 of the reservoir 10, as shown in FIG. 6.

Said open distal end 12 of the reservoir 10 is for receiving said plunger 30. Said plunger 30 is adapted to connect to said distal end 22 of the piston 20, advantageously by means of an outside screw thread (not shown) formed on said plunger 30 and co-operating with an inside screw thread 27 formed in said piston 20. The piston 20 is thus advantageously screwed onto said plunger 30. It is also possible to envisage other means for fastening the piston 20 on the plunger 30.

In the invention, said proximal end walls 14 and 24 of the reservoir 10 and of the piston 20 are conical. Said proximal end wall 14 of the reservoir 10 forms an angle α, and said proximal end wall 24 of the piston 20 forms an angle β. According to an aspect of the invention, the difference between the angle α and the angle β lies in the range 3° to 5°, and is preferably equal to 4°. The fact that this difference is greater than or equal to 3° guarantees that, after actuation, the tip 25 of the piston does indeed close the through opening 15 in sealed manner and without any risk of reflux. The fact that this difference is less than or equal to 5°, and is preferably equal to 4°, serves to limit the dead volume that remains between the piston and the reservoir after actuation, and thus guarantees expulsion of nearly all of the fluid that was contained in said reservoir 10 prior to actuation.

In a preferred embodiment, the angle α is equal to 145° (±1°) and the angle β is equal to 141° (±1°).

In an advantageous aspect of the present invention, the piston 20 includes non-stick means for preventing two or more pistons 20 sticking together while the pistons are in storage, prior to being assembled in syringe assembles. This storage generally takes place by placing a plurality of pistons in a common package. If the pistons become stuck to one another during this storage stage, it can be difficult to assemble syringe assemblies on assembly lines. It is therefore desirable to be able to avoid any risk of the pistons sticking together before assembly in syringe assemblies, where a respective piston is inserted in a respective reservoir.

In a first variant of the non-stick means, shown in particular in FIG. 8, the outside surface of said proximal end wall 24 of the piston 20, which is the surface of the piston that comes into contact with the fluid, has an annular zone 240 of surface that is modified, in particular by graining. This graining may be made in any appropriate manner, e.g. by physical modification of the mold in said outside surface of said proximal end wall 24 of the piston 20, in particular by electroerosion or by spraying abrasive media. Said modified annular zone 240 does not include the tip 25 of said proximal end wall 24 of the piston 20, which tip, after actuation, co-operates in leaktight manner with said through opening 15 in said proximal end wall 14 of the reservoir 10. The modified annular zone 240 also does not include the radially outer edge of said proximal end wall 14 of the reservoir 10, which outer edge co-operates in leaktight manner with said cylindrical side wall 11 of the reservoir 10. This annular zone 240 serves to avoid any risk of pistons sticking together during storage of said pistons prior to being assembled in syringe assemblies. Specifically, and as can be seen in FIG. 8, the presence of the annular zone 240 serves to avoid any suction cup effect occurring as might otherwise happen when two pistons become stuck together with the proximal end wall 24 of one piston becoming stuck in leaktight manner against the distal end 22 of another piston. The modified annular surface zone 240 prevents any sealing in that zone, and thus prevents any risk of the pistons sticking together. Unlike the projecting profiles in the state of the art, which would prevent the through opening 15 of the reservoir 10 being closed in leaktight manner by the axial tip 25 of the piston 20 after actuation, the presence of the annular zone 240 has no impact on said leaktight co-operation, in spite of the small difference between the angles α and β. Whereas projecting profiles require a difference of angle of at least 6°, or even 10°, as in the above-mentioned prior art documents, thereby having a negative impact of increasing the dead volume, the annular zone 240 serves to avoid two pistons sticking together during storage, while minimizing said dead volume with a very small difference of angle.

In a second variant of the non-stick means, shown in FIG. 7, said distal end 22 of the piston 20 has a distal end surface provided with at least one axially projecting profile 220. In the figures, and in particular in FIG. 4, three axially projecting profiles 220 are shown. These axially projecting profiles 220 also serve to avoid any risk of sticking between two pistons, as shown in FIG. 7. The presence of said axially projecting profiles 220 prevents any leaktight co-operation between the proximal end wall 24 of one piston and the distal end 22 of another piston.

Advantageously, the piston 20 may have both an annular zone 240 on its proximal end wall 24 and axially projecting profiles on its distal end 22.

Although the present invention is described with reference to advantageous embodiments thereof, it should be understood that the invention is not limited to these embodiments, but on the contrary any useful modifications may be made by the person skilled in the art without going beyond the ambit of the present invention as defined by the accompanying claims.

1. An anti-reflux syringe assembly comprising:
a reservoir for containing a fluid, said reservoir having a cylindrical side wall, an open distal end, and a proximal end having a proximal end wall provided at its axial center with a through opening; and
a piston slidable in leaktight manner in said reservoir, said piston having a distal end adapted to connect to a plunger passing through said open distal end of the reservoir, and a proximal end having a proximal end wall;
the assembly being characterized in that said proximal end walls of the reservoir and of the piston are conical, said proximal end wall of the reservoir forming an angle α and said proximal end wall of the piston forming an angle β, with:
3°<α−β<5°

2. An assembly according to claim 1, wherein:
α−β=4°

3. An assembly according to claim 1, wherein:
α=145° (35 1°)
and
β=141° (±1°)

4. An assembly according to claim 3, wherein the outside surface of said proximal end wall of the piston in contact with the fluid has an annular zone of surface that is modified, in particular by graining.

5. An assembly according to claim 4, wherein said modified annular zone does not include the tip of said proximal end wall of the piston, which tip, after actuation, co-operates in leaktight manner with said through opening in said proximal end wall of the reservoir.

6. An assembly according to claim 5, wherein the distal end of the piston has a distal end surface provided with at least one axially projecting profile.

7. An assembly according to claim 6, wherein said piston has an inside screw thread adapted to screw onto a corresponding outside screw thread provided on said plunger, said piston being screwed on said plunger.
